(19) United States
(12) Patent Application Publication
(10) Pub. No.: US 2025/0360153 A1
(43) Pub. Date: Nov. 27, 2025

(54) PHARMACEUTICAL COMPOSITION FOR PREVENTING OR TREATING ACNE

(71) Applicant: SEUNG CHANG LEE, Seoul (KR)

(72) Inventor: SEUNG CHANG LEE, Seoul (KR)

(21) Appl. No.: 18/871,608

(22) PCT Filed: Jun. 8, 2023
(86) PCT No.: PCT/KR2023/007879

§ 371 Date: Dec. 4, 2024

(30) Foreign Application Priority Data
Jun. 14, 2022 (KR) 10-2022-0071968
Jun. 7, 2023 (KR) 10-2023-0072916

(51) Int. Cl. A61K 31/7056; A61K 9/06; A61K 31/4745; A61K 31/65; A61K 33/06; A61K 33/24; A61K 47/10; A61K 47/36; A61P 17/10

(52) U.S. Cl. CPC A61K 31/7056; A61K 9/06; A61K 31/4745; A61K 31/65; A61K 33/06; A61K 33/24; A61K 47/10; A61K 47/36; A61P 17/10

(57) ABSTRACT

A composition for preventing or treating acne includes antibiotics, bentonite, kaolin clay, corn starch, agar, titanium dioxide (TiO2), and talc, thereby making it possible to form a formulation that not only applies smoothly to the skin and does not flow down immediately after application to the skin, but also adheres well to the skin even after time has passed so as to be maintained well therein, and exhibiting excellent acne prevention or treatment effects.

### BACKGROUND

### 1. Technical Field

The present invention relates to a pharmaceutical composition for preventing or treating acne.

### 2. Background Art

Acne refers to inflammation of many hair follicles on the face of adolescent men and women, especially on the cheeks and forehead. The acne may appear on the chest or back, and may occur even around the age of 40. At first, the hair follicles are filled with cortex and keratin, forming a yellowish-white lump, and sometimes the apex may turn black. At this time, if squeezing the acne, something shaped like cheese will come out. Then, inflammation occurs and redness develops around this area. When bacteria infect it, the area becomes purulent and turns yellow. The healed spot leaves a crater-shaped trace. Usually, these four types are mixed. The cause of these conditions is often bacterial infection of sebum secreted excessively due to congenital predisposition and the action of sex hormones, especially male hormones. In women over the age of 22, it often worsens just before menstruation. The cause of acne is not yet clearly known, but it can generally be summarized as increased sebum secretion, skin keratinization and peroxidation of lipid components, proliferation of acne-causing bacteria, and induction and worsening of inflammation. One of the important characteristics related to the onset of the acne is an increase in sebum secretion, which is known to be closely related to androgen, a male hormone. When male hormones are secreted the normal level or more, the function of the sebaceous glands is increased, and the excess sebum thus produced stagnates in the hair follicles due to keratinization of the hair follicle wall, thereby resulting in the formation of comedones. The sebum accumulated in the hair follicle, which could not be discharged to the outside, blocks the pore and prevents the inflow of external air, thus creating an environment in which the anaerobic bacterium Propionibacterium acnes can proliferate smoothly inside the hair follicle. Propionibacterium acnes secrete lipolytic enzymes and chemical factors to produce free fatty acids, triggers an immune response of white blood cells, destroys the hair follicle wall, and induces the contents to leak into the dermis, hence causing an inflammatory reaction. Therefore, the general treatment method for acne would be summarized in four cases: suppressing excessive sebum production, inhibiting keratinization of the hair follicle wall, suppressing the proliferation of Propionibacterium acnes, and minimizing or inhibiting the inflammatory response.

Diverse microorganisms exist on human skin. These microorganisms cause various skin diseases. In addition to Staphylococcus aureus, which causes purulence on the skin, there are skin normal bacteria such as Staphylococcus epidermis and Propionibacterium acnes, etc. These bacteria decompose sebum and sweat secreted on the skin and generate odor, in addition, the decomposition products irritate the skin and cause inflammation.

Meanwhile, topical liquids which are applied topically to acne areas on the skin are commonly used to improve the acne. Since such topical liquids are applied topically, they have an advantage of being relatively safer compared to oral agents that affect systemic hormones. However, the topical liquids are generally manufactured in a gel or solution type, which has a problem of causing dryness in the applied area. Therefore, there is a need for the development of a formulation that adheres well to the skin, and does not cause significant dryness while having similar or superior acne treatment effects to the typical topical liquids.

### SUMMARY

An object of the present invention is to provide a pharmaceutical composition for preventing or treating acne.
1. A pharmaceutical composition for preventing or treating acne, including antibiotics, bentonite, kaolin clay, corn starch, agar, titanium dioxide (TiO₂) and talc.
2. The pharmaceutical composition for preventing or treating acne according to the above 1, wherein a weight ratio of the antibiotics and the bentonite is 1:5 to 25.
3. The pharmaceutical composition for preventing or treating acne according to the above 1, wherein the composition includes 500 to 2500 parts by weight of the bentonite, 100 to 500 parts by weight of the kaolin clay, 20 to 200 parts by weight of the corn starch, 5 to 20 parts by weight of agar, 100 to 400 parts by weight of the titanium dioxide, and 15 to 50 parts by weight of the talc, based on 100 parts by weight of the antibiotics.
4. The pharmaceutical composition for preventing or treating acne according to the above 1, further including glycerin.
5. The pharmaceutical composition for preventing or treating acne according to the above 1, wherein the antibiotics include one selected from the group consisting of clindamycin, tetracycline, nadifloxacin, erythromycin, azithromycin, roxithromycin, minocycline and doxycycline.
6. The pharmaceutical composition for preventing or treating acne according to the above 1, wherein the composition is solid or semi-solid.
7. The pharmaceutical composition for preventing or treating acne according to the above 1, wherein the formulation of the pharmaceutical composition is selected from the group consisting of ointments, creams and pastes, and cataplasma agents.
8. A method for manufacturing a pharmaceutical composition for prevention or treatment of acne, which includes: (a) adding a second composition including agar to a first composition including corn starch to prepare a mixture;
(b) adding a third composition including an antibiotic to the mixture obtained in the step (a); and
(c) adding kaolin clay, titanium dioxide (TiO₂), talc and bentonite to the mixture obtained in the step (b).
9. The method according to the above 8, wherein the second composition further includes glycerin.
10. The method according to the above 8, wherein the third composition is dissolved by heating.

The pharmaceutical composition for preventing or treating acne of the present invention makes it is possible to form a formulation that not only applies smoothly to the skin and does not flow down immediately after application to the skin, but also adheres well to the skin even after time has passed, such that the antibiotic effect lasts for a long time thus to exhibit excellent acne preventive or therapeutic effects.

### DETAILED DESCRIPTION

Hereinafter, the present invention will be described.

The present invention provides a pharmaceutical composition for preventing or treating acne.

The pharmaceutical composition may include antibiotics, bentonite, kaolin clay, corn starch, agar, titanium dioxide (TiO₂), and talc.

The present inventors have found that the pharmaceutical composition, which includes the above-described components, not only applies smoothly to the skin and does not flow down immediately after application to the skin, but also adheres well to the skin even after time has passed, and has a formulation advantage of excellent moisturizing and skin soothing effects, in addition, that a stable formulation could be formed without significant differences depending on the types of antibiotics included in the pharmaceutical composition. Further, the present inventors have confirmed that the pharmaceutical composition exhibits excellent acne prevention or treatment effects as well as the above-described formulation advantages.

The antibiotics are not limited as long as they can improve acne, specifically may include one selected from the group consisting of clindamycin, tetracycline, nadifloxacin, erythromycin, azithromycin, roxithromycin, minocycline and doxycycline.

The bentonite is a natural mineral formed through hydrothermal alteration of rocks due to volcanic activity and is a clay mineral containing montmorillonite as a major ingredient thereof. The bentonite may be used as a thickener to give the pharmaceutical composition an appropriate viscosity for application to the skin.

In one embodiment, a weight ratio of the antibiotics and the bentonite may be 1:5 to 25, 1:5 to 20, 1:7 to 20, 1:10 to 20, or 1:10 to 15.

In one embodiment, when the pharmaceutical composition includes a large amount of bentonite compared to the antibiotics, it may have a formulation suitable for application to the skin and may be less likely to cause dryness when applied to the skin.

The kaolin clay is a naturally produced hydrous aluminum silicate, which is a natural mineral similar to clay that exists in white or milky white powder form. The kaolin clay is a porous plate-shaped powder that can absorb a large amount of water and oil, thereby improving the adhesion, absorption and oil absorption of the pharmaceutical composition.

In one embodiment, the kaolin clay content may be 100 to 500 parts by weight (“wt. parts”), 150 to 400 wt. parts, 200 to 300 wt. parts, or 220 to 270 wt. parts, based on 100 wt. parts of the antibiotics. In this case, the kaolin clay is sufficiently dissolved such that the pharmaceutical composition may exhibit superior acne treatment effects without impairing the formulation stability of the pharmaceutical composition.

The corn is known to prevent skin aging and is effective in improving blemishes and fine wrinkles. Further, the corn is rich in ingredients such as protein, fiber, vitamins, and minerals, etc., therefore, is effective in moisturizing the skin and preventing skin eczema or allergies.

In one embodiment, the corn starch content is 20 to 200 wt. parts, 40 to 160 wt. parts, 60 to 140 wt. parts, 70 to 130 wt. parts, or 80 to 120 wt. parts based on 100 wt. parts of the antibiotic. In this case, the pharmaceutical composition may exhibit superior acne treatment effects without impairing the stability of the pharmaceutical composition due to the corn starch.

The agar is made by boiling and cooling agar-agars and drying them through freeze-dehydration or pressing-dehydration. The agar is dissolved in a high-temperature aqueous phase, and when cooled again, forms a gel state, and is used as a thickener.

In one embodiment, the agar content may be 5 to 20 wt. parts, 6 to 16 wt. parts, 7 to 14 wt. parts, or 8 to 12 wt. parts, based on 100 wt. parts of the antibiotic. In this case, the agar may be used to form an appropriate formulation without affecting acne-improving effects of the pharmaceutical composition.

In one embodiment, the titanium dioxide content may be 100 to 400 wt. parts, 150 to 350 wt. parts, 200 to 300 wt. parts, or 230 to 280 wt. parts, based on 100 wt. parts of the antibiotics.

The talc is a natural hydrous magnesium silicate mineral containing a small amount of aluminum silicate and may prevent agglomeration or coagulation of the pharmaceutical composition, and improve application uniformity. The pharmaceutical composition including the talc may improve ease of application, skin spreadability, texture, etc.

In one embodiment, the talc content may be 15 to 50 wt. parts, 20 to 45 wt. parts, 30 to 40 wt. parts, or 32 to 37 wt. parts, based on 100 wt. parts of the antibiotic. In this case, the pharmaceutical composition may exhibit better acne treatment effects without deteriorating the action and function of other ingredients included in the pharmaceutical composition.

In one embodiment, the bentonite content may be 500 to 2500 wt. parts, the kaolin clay content may be 100 to 500 wt. parts, the corn starch content may be 20 to 200 wt. parts, the agar content may be 5 to 20 wt. parts, the titanium dioxide content may be 100 to 400 wt. parts, and the talc content may be 15 to 50 wt. parts, based on 100 parts by weight of the antibiotics. In this case, each ingredient included in the pharmaceutical composition allows the pharmaceutical composition to have excellent skin applicability and viscosity without impairing the effects of other ingredients, and further allows the active ingredient for improving acne to remain on the skin for a long time, such that it may have better acne treatment effects.

In one embodiment, the pharmaceutical composition may further include glycerin. The glycerin acts to retain moisture, as well as increases the flexibility of the skin. Further, it may restore the skin's regeneration cycle and help to maintain the function of the skin barrier.

In one embodiment, the pharmaceutical composition may be manufactured by a method including: (a) adding a second composition including agar to a first composition including corn starch to prepare a mixture; (b) adding a third composition including an antibiotic to the mixture obtained in the step (a); and (c) adding kaolin clay, titanium dioxide, talc and bentonite to the mixture obtained in the step (b).

The pharmaceutical composition may be solid or semi-solid. A formulation of the pharmaceutical composition may be selected from the group consisting of ointment, cream and paste. In this case, when the pharmaceutical composition is applied to the skin, it may adhere closely to the skin and remain well for a long time compared to the case where the pharmaceutical composition is in a liquid form, thereby attaining less dryness and providing excellent acne prevention or treatment effects.

The pharmaceutical composition may be used as a topical skin preparation. For example, the pharmaceutical composition may be used by applying it to a local area of the skin. For example, the pharmaceutical composition may be used by applying it to the area where acne is formed on the skin. Thereby, the acne at the area where the pharmaceutical composition is applied may be treated.

The term “prevention” refers to prophylactic measures that result in any extent of reduction in probability of development of pathogenic conditions to be prevented or reoccurring or recurring conditions, including total prevention as well as slight, substantial or significant reduction of probability of development or reoccurrence of pathogenic conditions, wherein the reduction extent of probability may be at least a slight reduction.

The term “treatment” refers to treatment that results in beneficial effects on a subject or patient suffering from the pathogenic condition being treated, including cure as well as any extent of relief including mild relief, substantial relief, significant relief, wherein the relief extent may be at least mild relief.

The present invention provides a method for manufacturing a pharmaceutical composition for preventing or treating acne.

The method for manufacturing a pharmaceutical composition may include the following steps of:
(a) preparing a mixture by adding a second composition including agar to a first composition including corn starch;
(b) adding a third composition including an antibiotic to the mixture obtained in the step (a); and
(c) adding kaolin clay, titanium dioxide (TiO₂), talc and bentonite to the mixture obtained in the step (b).

The corn starch, agar, antibiotics, kaolin clay, titanium dioxide, talc, bentonite, their contents, the term “prevention” and the term “treatment” may be within the above-described range, but they are not limited thereto.

In one embodiment, the second composition may further include at least one selected from the group consisting of glycerin and purified water.

In one embodiment, the second composition may be prepared by adding purified water to glycerin and agar, and then dispersing it at room temperature.

The third composition may be dissolved by heating. Temperature conditions during the heating and dissolution may be 25 to 60° C., 30 to 50° C., 35 to 45° C., or 37 to 42° C.

The step (c) may be performed by sequentially injecting the kaolin clay, titanium dioxide, talc and bentonite into a container.

In one embodiment, the method for manufacturing a pharmaceutical composition may include the following steps (i) to (iv) of:
(i) injecting corn starch into a container and stirring and dissolving at room temperature;
(ii) adding purified water to glycerin and agar, dispersing it at room temperature, and then injecting it into the container;
(iii) injecting antibiotics into the container and stirring uniformly; and
(iv) sequentially injecting kaolin clay, titanium dioxide, talc and bentonite into the container.

The pharmaceutical composition prepared by the above manufacturing method may form and maintain a stable formulation, and when applied to the skin, it adheres closely to the skin and remains well for a long time compared to the liquid pharmaceutical composition, thereby attaining less dryness and excellent acne prevention or treatment effects.

### EXAMPLE

### 1. Method of Manufacturing a Topical Pack for Improving Acne

### <1-1> Preparation of First Mixture

Corn starch was injected into a container and stirred and dissolved at room temperature to prepare a first mixture.

### <1-2> Preparation of Second Mixture

Purified water was added to glycerin and agar, dispersed at room temperature, and then injected into the container to prepare a second mixture.

### <1-3> Preparation of Third Mixture

Antibiotics (clindamycin, tetracycline, or nadifloxacin) were dissolved by heating at 40° C., then injected into the container and stirred uniformly to prepare a third mixture.

### <1-4> Pack Manufacturing

Kaolin, talc, titanium dioxide (TiO₂) and bentonite were sequentially added to the container and thoroughly stirred for 45 minutes to prepare a pack. The titanium dioxide was used as a solution with a concentration of 50 wt. % using propylene glycol as a solvent.

### 2. Manufactured Topical Pack for Acne Treatment (Semi-Solid Product)

Topical application packs for treating acne of Examples 1 to 17 and Comparative Examples 1 to 6 were prepared through the above manufacturing method.

### (1) Pack Manufacturing Conditions of Examples 1 to 13 and Comparative Examples 1 to 6

Examples 1 to 17 and Comparative Examples ito 6 were prepared using bentonite (Bentonite 670), kaolin clay, corn starch, agar, titanium dioxide (TiO₂), talc, and purified water at the contents (unit: kg) shown in Table 1 below.

The types of antibiotics used in the examples and comparative examples are as follows: clindamycin (Examples 1 to 11 and Comparative Examples 1 to 6), tetracycline (Examples 12 to 14), and nadifloxacin (Examples 15 to 17).

TABLE 1

| | | | Kaolin | Corn | | Titanium | |
|---|---|---|---|---|---|---|---|
| (Unit: kg) | Antibiotics | Bentonite | clay | starch | Agar | dioxide | Talc |
| Example 1 | 0.2 | 2.5 | 0.5 | 0.2 | 0.02 | 0.52 | 0.07 |
| Example 2 | 0.2 | 1 | 0.5 | 0.2 | 0.02 | 0.52 | 0.07 |
| Example 3 | 0.2 | 5 | 0.5 | 0.2 | 0.02 | 0.52 | 0.07 |
| Example 4 | 0.2 | 2.5 | 0.2 | 0.2 | 0.02 | 0.52 | 0.07 |
| Example 5 | 0.2 | 2.5 | 1 | 0.2 | 0.02 | 0.52 | 0.07 |
| Example 6 | 0.2 | 2.5 | 0.5 | 0.04 | 0.02 | 0.52 | 0.07 |
| Example 7 | 0.2 | 2.5 | 0.5 | 0.4 | 0.02 | 0.52 | 0.07 |
| Example 8 | 0.2 | 2.5 | 0.5 | 0.2 | 0.01 | 0.52 | 0.07 |
| Example 9 | 0.2 | 2.5 | 0.5 | 0.2 | 0.04 | 0.52 | 0.07 |
| Example 10 | 0.2 | 2.5 | 0.5 | 0.2 | 0.02 | 0.2 | 0.07 |
| Example 11 | 0.2 | 2.5 | 0.5 | 0.2 | 0.02 | 0.8 | 0.07 |
| Example 12 | 0.2 | 2.5 | 0.5 | 0.2 | 0.02 | 0.52 | 0.07 |
| Example 13 | 0.2 | 1 | 0.5 | 0.2 | 0.02 | 0.52 | 0.07 |
| Example 14 | 0.2 | 5 | 0.5 | 0.2 | 0.02 | 0.52 | 0.07 |
| Example 15 | 0.2 | 2.5 | 0.5 | 0.2 | 0.02 | 0.52 | 0.07 |
| Example 16 | 0.2 | 1 | 0.5 | 0.2 | 0.02 | 0.52 | 0.07 |
| Example 17 | 0.2 | 5 | 0.5 | 0.2 | 0.02 | 0.52 | 0.07 |
| Comparative | 0.2 | — | 0.5 | 0.2 | 0.02 | 0.52 | 0.07 |
| Example 1 | | | | | | | |
| Comparative | 0.2 | 2.5 | — | 0.2 | 0.02 | 0.52 | 0.07 |
| Example 2 | | | | | | | |
| Comparative | 0.2 | 2.5 | 0.5 | — | 0.02 | 0.52 | 0.07 |
| Example 3 | | | | | | | |
| Comparative | 0.2 | 2.5 | 0.5 | 0.2 | — | 0.52 | 0.07 |
| Example 4 | | | | | | | |
| Comparative | 0.2 | 2.5 | 0.5 | 0.2 | 0.02 | — | 0.07 |
| Example 5 | | | | | | | |
| Comparative | 0.2 | 2.5 | 0.5 | 0.2 | 0.02 | 0.52 | — |
| Example 6 | | | | | | | |

— Purified water: Balance

### Experimental Example

### 1. Assessment of Formulation Convenience

Formulation convenience was assessed by observing spreadability, adhesion, and retention in the packs of Examples 1 to 17 and Comparative Examples 1 to 6.

A panel consisting of 50 adult men and women in their 20s to 40s who were suitable for the test purpose through medical history and skin condition diagnosis was asked to apply the packs of Examples 1 to 17 and Comparative Examples 1 to 6 to the skin.

The spreadability was evaluated to determine whether it spreads smoothly when applied to the skin. The adhesion was evaluated to ensure that it adheres well to the skin without flowing down immediately after application. The retention was evaluated to determine whether the pack was adhered to the skin and remained well even 3 hours after applying it to the skin. Each score ranged from 1 to 5, with 1 of ‘very poor,’ 3 of ‘average,’ and 5 of ‘very good.’ Experimental results are indicated as an average score of 50 people in Table 2 below.

TABLE 2

| Division | Spreadability | Adhesion | Retention |
|---|---|---|---|
| Example 1 | 4.96 | 4.98 | 4.98 |
| Example 2 | 4.92 | 4.86 | 4.88 |
| Example 3 | 4.84 | 4.92 | 4.94 |
| Example 4 | 4.96 | 4.94 | 4.94 |
| Example 5 | 4.92 | 4.94 | 4.94 |
| Example 6 | 4.94 | 4.92 | 4.92 |
| Example 7 | 4.92 | 4.94 | 4.94 |
| Example 8 | 4.96 | 4.96 | 4.96 |
| Example 9 | 4.94 | 4.96 | 4.94 |
| Example 10 | 4.94 | 4.92 | 4.92 |
| Example 11 | 4.94 | 4.94 | 4.94 |
| Example 12 | 4.96 | 4.98 | 4.98 |
| Example 13 | 4.92 | 4.86 | 4.88 |
| Example 14 | 4.86 | 4.92 | 4.92 |
| Example 15 | 4.96 | 4.98 | 4.98 |
| Example 16 | 4.92 | 4.88 | 4.88 |
| Example 17 | 4.82 | 4.92 | 4.90 |
| Comparative | 1.02 | 1.14 | 1.42 |
| Example 1 | | | |
| Comparative | 2.42 | 1.84 | 1.76 |
| Example 2 | | | |
| Comparative | 1.76 | 2.46 | 2.42 |
| Example 3 | | | |
| Comparative | 3.02 | 2.78 | 2.86 |
| Example 4 | | | |
| Comparative | 2.98 | 3.02 | 2.96 |
| Example 5 | | | |
| Comparative | 2.16 | 2.14 | 2.06 |
| Example 6 | | | |

As shown in Table 2, it can be seen that Examples 1 to 17 according to the present invention are superior to Comparative Examples 1 to 6 in terms of the spreadability, adhesion and retention.

### 2. Antibacterial Activity Test Against Acne Bacteria

Propionibacterium acnes (ATCC 6919: medium-BHI broth), a strain that causes acne, were tested for antibacterial activity using a known topical liquid for treating acne (active ingredient: clindamycin) and the packs of Examples 1 to 17 and Comparative Examples 1 to 6.

The antibacterial activity test method is as follows:
1) Propionibacterium acnes was inoculated into BHI broth and a test bacterial solution was prepared using the culture medium cultured anaerobically.
2) 0.15 ml of the test bacterial solution was added to 15 ml of BHI broth (pH 6.8) or LB broth (pH 4.5) and mixed well, which was used as a diluted solution.
3) The packs of Examples 1 to 17 and Comparative Examples 1 to 6 as well as known topical liquid for treating acne were directly used as samples.
4) The sample entered row No. 1 of a 96 well plate to match the starting concentration, followed by adding the dilution solution to have a total volume of 200 μl.
5) After mixing the mixture in row No. 1 above, 100 μl of the mixture was taken and added to row No. 2, mixed well, followed by executing double dilution such that 100 μl was taken again and then added to row No. 3.
6) After stationary incubation at 32° C. for 24 and 48 hours, the presence or absence of bacterial growth was determined based on the extent of suspension. The minimum concentration without bacterial growth is determined as a minimum inhibitory concentration (MIC) value. If the mixed solution is opaque and it is difficult to determine the presence or absence of bacterial growth, it was checked through microscopic observation.

### 3. Evaluation of Acne Improvement and Presence or Absence of Irritation

A panel consisting of 50 adult men and women in their 20s to 40s who were suitable for the test purpose through medical history and skin condition diagnosis was asked to use a known topical liquid for treating acne (active ingredient: clindamycin), as well as the packs of Examples 1 to 17 and Comparative Examples 1 to 6 for one month.

The acne improvement scale ranged from 1 to 5, with 1 of ‘very poor,’ 3 of ‘average,’ and 5 of ‘very good.’ The experimental results are expressed as an average score of 50 people in Table 5 below. The presence or absence of skin irritation was expressed as (the number of people showing irritation reaction)/(the total number of testers).

TABLE 3

| | Acne improvement | | Presence or |
|---|---|---|---|
| | Inflammatory acne | Comedonal acne | absence of |
| Division | improvement | improvement | irritation |
| Known topical | 4.18 | 4.08 | 20/50 |
| liquid | | | |
| Example 1 | 4.58 | 4.50 | 0/50 |
| Example 2 | 4.52 | 4.46 | 0/50 |
| Example 3 | 4.64 | 4.62 | 0/50 |
| Example 4 | 4.56 | 4.48 | 0/50 |
| Example 5 | 4.60 | 4.52 | 0/50 |
| Example 6 | 4.56 | 4.48 | 0/50 |
| Example 7 | 4.56 | 4.46 | 0/50 |
| Example 8 | 4.58 | 4.50 | 0/50 |
| Example 9 | 4.54 | 4.44 | 0/50 |
| Example 10 | 4.52 | 4.46 | 0/50 |
| Example 11 | 4.54 | 4.44 | 0/50 |
| Example 12 | 4.54 | 4.42 | 0/50 |
| Example 13 | 4.52 | 4.40 | 0/50 |
| Example 14 | 4.56 | 4.42 | 0/50 |
| Example 15 | 4.52 | 4.42 | 0/50 |
| Example 16 | 4.52 | 4.40 | 0/50 |
| Example 17 | 4.54 | 4.44 | 0/50 |
| Comparative | 2.80 | 2.62 | 1/50 |
| Example 1 | | | |
| Comparative | 3.34 | 3.12 | 0/50 |
| Example 2 | | | |
| Comparative | 3.42 | 3.34 | 0/50 |
| Example 3 | | | |
| Comparative | 3.48 | 3.36 | 0/50 |
| Example 4 | | | |
| Comparative | 3.44 | 3.34 | 0/50 |
| Example 5 | | | |
| Comparative | 3.80 | 3.54 | 0/50 |
| Example 6 | | | |

As shown in Table 2, it can be seen that Examples 1 to 17 according to the present invention showed excellent acne improvement effects compared to Comparative Examples 1 to 6.

### 4. Evaluation of Moisturization and Skin Soothing Effect

A panel consisting of 50 adult men and women in their 20s to 40s who were suitable for the test purpose through medical history and skin condition diagnosis was asked to use a known topical liquid for treating acne (active ingredient: clindamycin), as well as the packs of Examples 1 to 17 and Comparative Examples 1 to 6, so as to assess moisturization and skin soothing effects.

After taking an appropriate amount of each of the known topical liquid for treating acne (active ingredient: clindamycin) and the packs of Examples 1 to 17 and Comparative Examples 1 to 6 and applying the same to acne area in a thin layer before going to bed, it was washed off from the face the next morning. According to the panel's opinion, the extent of improvement was evaluated using a 5-level discrimination method (1: very poor, 2: poor, 3: average, 4: excellent, 5: very good), and the average value was derived and results thereof are shown in Table 4 below.

TABLE 4

| Division | Moisturization | Skin soothing effect |
|---|---|---|
| Known topical liquid | 3.20 | 3.88 |
| Example 1 | 4.78 | 4.64 |
| Example 2 | 4.64 | 4.58 |
| Example 3 | 4.80 | 4.70 |
| Example 4 | 4.58 | 4.52 |
| Example 5 | 4.60 | 4.60 |
| Example 6 | 4.62 | 4.60 |
| Example 7 | 4.78 | 4.64 |
| Example 8 | 4.76 | 4.62 |
| Example 9 | 4.77 | 4.66 |
| Example 10 | 4.74 | 4.72 |
| Example 11 | 4.72 | 4.70 |
| Example 12 | 4.76 | 4.74 |
| Example 13 | 4.76 | 4.74 |
| Example 14 | 4.76 | 4.74 |
| Example 15 | 4.76 | 4.72 |
| Example 16 | 4.76 | 4.74 |
| Example 17 | 4.76 | 4.76 |
| Comparative Example 1 | 3.34 | 3.54 |
| Comparative Example 2 | 3.76 | 3.72 |
| Comparative Example 3 | 3.44 | 3.46 |
| Comparative Example 4 | 4.32 | 4.30 |
| Comparative Example 5 | 4.24 | 4.22 |
| Comparative Example 6 | 3.44 | 3.66 |

Further, 20 minutes after washing the face, the skin moisture content was measured using a skin moisture meter (using the Moisture Sense device from Moritex, Japan), and the final skin moisture content was derived by averaging the measurements of each of the 50 panelists, and results thereof are shown in Table 7 below.

TABLE 5

| Division | Skin moisture content (%) |
|---|---|
| Known topical liquid | 19.8 |
| Example 1 | 25.3 |
| Example 2 | 25.8 |
| Example 3 | 25.4 |
| Example 4 | 25.4 |
| Example 5 | 25.2 |
| Example 6 | 25.2 |
| Example 7 | 25.5 |
| Example 8 | 25.2 |
| Example 9 | 25.5 |
| Example 10 | 256.3 |
| Example 11 | 25.1 |
| Example 12 | 25.4 |
| Example 13 | 25.4 |
| Example 14 | 25.3 |
| Example 15 | 25.4 |
| Example 16 | 25.2 |
| Example 17 | 25.3 |
| Comparative Example 1 | 18.7 |
| Comparative Example 2 | 19.0 |
| Comparative Example 3 | 18.5 |
| Comparative Example 4 | 19.2 |
| Comparative Example 5 | 19.1 |
| Comparative Example 6 | 18.5 |

As shown in Tables 4 and 5, it can be seen that Examples 1 to 17 according to the present invention have superior moisturization and skin soothing effects compared to Comparative Examples 1 to 6.

The composition of the present invention makes it is possible to form a formulation that not only applies smoothly to the skin and does not flow down immediately after application to the skin, but also adheres well to the skin even after time has passed, such that the antibiotic effect lasts for a long time thus to exhibit excellent acne preventive or therapeutic effects. Therefore, it can be used as a pharmaceutical composition for preventing or treating acne.

1: A pharmaceutical composition for preventing or treating acne, comprising antibiotics, bentonite, kaolin clay, corn starch, agar, titanium dioxide (TiO₂) and talc.

2: The pharmaceutical composition for preventing or treating acne according to claim 1, wherein a weight ratio of the antibiotics and the bentonite is 1:5 to 25.

3: The pharmaceutical composition for preventing or treating acne according to claim 1, wherein the composition comprises 500 to 2500 parts by weight of the bentonite, 100 to 500 parts by weight of the kaolin clay, 20 to 200 parts by weight of the corn starch, 5 to 20 parts by weight of agar, 100 to 400 parts by weight of the titanium dioxide, and 15 to 50 parts by weight of the talc, based on 100 parts by weight of the antibiotics.

4: The pharmaceutical composition for preventing or treating acne according to claim 1, further comprising glycerin.

5: The pharmaceutical composition for preventing or treating acne according to claim 1, wherein the antibiotics include one selected from the group consisting of clindamycin, tetracycline, nadifloxacin, erythromycin, azithromycin, roxithromycin, minocycline and doxycycline.

6: The pharmaceutical composition for preventing or treating acne according to claim 1, wherein the composition is solid or semi-solid.

7: The pharmaceutical composition for preventing or treating acne according to claim 1, wherein the formulation of the pharmaceutical composition is selected from the group consisting of ointments, creams and pastes, and cataplasma agents.

8: A method for manufacturing a pharmaceutical composition for prevention or treatment of acne, the method comprising:
(a) adding a second composition including agar to a first composition including corn starch to prepare a mixture;
(b) adding a third composition including an antibiotic to the mixture obtained in the step (a); and
(c) adding kaolin clay, titanium dioxide (TiO₂), talc and bentonite to the mixture obtained in the step (b).

9: The method according to claim 8, wherein the second composition further includes glycerin.

10: The method according to claim 8, wherein the third composition is dissolved by heating.

11: A method for treating acne, the method comprising:
administering a composition comprising antibiotics, bentonite, kaolin clay, corn starch, agar, titanium dioxide (TiO₂) and talc to a subject in need thereof.

12: The method of claim 11, wherein a weight ratio of the antibiotics and the bentonite is 1:5 to 25.

13: The method of claim 11, wherein the composition comprises 500 to 2500 parts by weight of the bentonite, 100 to 500 parts by weight of the kaolin clay, 20 to 200 parts by weight of the corn starch, 5 to 20 parts by weight of agar, 100 to 400 parts by weight of the titanium dioxide, and 15 to 50 parts by weight of the talc, based on 100 parts by weight of the antibiotics.

14: The method of claim 11, wherein the composition further comprises glycerin.

15: The method of claim 11, wherein the antibiotics include one selected from the group consisting of clindamycin, tetracycline, nadifloxacin, erythromycin, azithromycin, roxithromycin, minocycline, doxycycline, and a combination thereof.

16: The method of claim 11, wherein the composition is solid or semi-solid.

17: The method of claim 11, wherein the formulation of the pharmaceutical composition is selected from the group consisting of ointments, creams and pastes, and cataplasma agents.
